(19) United States
(12) Patent Application Publication
(10) Pub. No.: US 2004/0023211 A1
(43) Pub. Date: Feb. 5, 2004

(54) System and method for optimizing drug theraphy for the treatment of diseases

(76) Inventors: Kees Groen, Ra Oudenbosch (NL); Paul Stoffels, Hoogstraten (BE)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/380,413

(22) PCT Filed: Sep. 17, 2001
(86) PCT No.: PCT/EP01/10971

(30) Foreign Application Priority Data
Sep. 15, 2000 (EP) 00203200

(51) Int. Cl. C12Q 1/70; G06F 19/00; G01N 33/48; G01N 33/50

(52) U.S. Cl. 435/5; 702/19

(57) ABSTRACT

The present invention concerns the optimization of hiv-1 therapy using the combination of a bioanalytical method, population pharmacokinetic models and phenotypic resistance testing.

This application claims priority benefit of European Patent Application No. 00/203,200.1, filed on Sep. 15, 2000, and U.S. Provisional Application No. 60/279,674 Mar. 30, 2001, the contents of which are expressly incorporated by reference herein.

### FIELD OF THE INVENTION

The present invention generally relates to the field of drug therapy, disease management, therapy monitoring and pharmacogenomics. In one embodiment, the present invention relates to systems and methods for designing or optimising a drug therapy for a patient in connection with the treatment of a disease. The present invention also provides an approach towards therapy design based on the integration of bio-analysis, pharmacological modelling and resistance testing.

### BACKGROUND OF THE INVENTION

Infectious agents including tuberculosis bacillus, human immunodificiency virus (HIV) and cell proliferative disorders have proven difficult to treat once affecting an individual. Efficacy of antiretroviral therapy is generally measured by a drop in viral load (concentration of viral RNA copies in the blood plasma), while antiretroviral therapy failure is generally reflected by an increase in viral load and/or the development of resistance to therapy. Likewise, anti-cancer drug treatments and therapies (i.e., chemotherapy, gene therapy, radiation, etc.) have proven effective against many malignancies and forms of cancer. However, many patients experience treatment failure, or reduced efficacy over time with many anti-cancer drugs and therapies. Such treatment failure may be due to a variety of causes, such as development of resistance to the particular drug via mutation or other process, progression of disease requiring an altered dosage regimen, patient noncompliance, sub-optimal pharmacokinetics, toxicity to a drug etc.

Intermittent blood level monitoring of drugs has been described in the literature as “therapeutic drug monitoring.” True therapeutic drug monitoring, in order to be accurate, would require constant, quantitative drug monitoring of blood concentrations in each individual patient for each administered drug. However, besides being prohibitively invasive and time consuming, such an approach suffers from various other practical shortcomings. Since such actual, constant blood level monitoring of all administered drugs is nearly impossible, some interval between samplings is required; different drugs may be administered at different times post-administration, leading to irregular sampled drug disposition curves.

Treatment success for many diseases, including cancer, infectious diseases and viral illnesses, is correlated with the use of optimal drug dosages, for both single drugs and for drugs in combination. Optimal dosages guarantee that the plasma drug concentration(s) remain well above the minimum effective concentrations (MECs) of all the administered drugs. Often, for example, the higher the MEC of a particular drug in a particular patient, the lower the disease sensitivity is to that particular drug, resulting in lower likelihood of effective treatment. The probability of treatment success depends on the fact that the MEC is drug-specific, and that for the same drug the MEC also varies across the patient population. Also, different drugs are more effective in some patients than in other patients due to inter-individual differences in pharmacokinetics. Individual patient characteristics also effect dosages, i.e., characteristics such as body size, gender, age, physical and pathophysiologic states, genetics, environment, and concurrent therapies. Therefore current day therapeutic monitorning services based on the sole determination of the concentration of a drug in a sample of a patient may have limited value.

Previous research has attempted to navigate effective dosages of drugs to challenge rapidly changing etiologic agents. While the broad approach of population pharmacokinetics (loosely defined as the change in time of the concentration or nature of therapeutic agent(s) in groups of patients having similar characteristics) is a technique of long standing (see T. M. Ludden, J. Clin. Pharmacol. 28:1059-1062 (1988)), it fails to take into account a large amount of inter-, and even intra-, patient variability, ultimately contributing to therapy failure. This is in part completed by the development of Bayesian parameter estimation in conjunction with population pharmacokinetics (Thomson & Whiting, Clin. Pharmacokinet; 1992, 22(6), 447-467). The combination of these parameters provides an approach to determine patient specific pharmacokinetic variables.

Another difficulty in the field of drug therapy is the development of drug resistance, which further stresses the need for individualized therapy. For example, continuous high level in vivo replication of retroviruses, particularly HIV, and the intrinsic error rate of the reverse transcriptase enzyme are major driving forces behind the generation of drug resistant virus variants. When sub-optimal drug dosages are applied as a pressure to this divergent and rapidly replicating virus population, variants with the appropriate mutations in their genome will escape drug inhibition and outgrow the wild-type, drug-susceptible viruses. Patients infected with such drug resistant strains are faced with ever narrowing therapeutic options. HIV drug resistance is an ever increasing problem, with an estimated 10 to 20% of patients in developed countries failing to respond to drug therapy in the first year of treatment and developing resistance to at least one drug.

Likewise, malignant cells, such as tumor cells, are subject to similar selection pressure by sub-optimal dosage therapy. Mutations accumulate over time, resulting in malignancies recalcitrant to drug therapy. One example of a specific mutational target is the tumor suppressor gene p53. The tumor suppressor gene p53, located on chromosome 17, is a key component of the body's anti-tumor defense (Soussi, T.; Ann. N.Y. Acad. Sci. 910:121-139 (2000); North, S. & Hainaut P.; Pathol. Biol. 48:255-270 (2000); Somasundaram, K.; Front. Biosci. 5: D424437 (2000); Tokino, T. & Nakamura, Y.; Crit. Rev. Oncol. Hematol. 33:1-6 (2000)). The p53 gene normally responds to DNA damage that might otherwise lead to cancer by arresting cell growth, initiating DNA repair, or sending cells into apoptosis (programmed cell death). When a p53 gene is mutated, however, the p53 gene, and the cells expressing it, become an etiological agents for cancer. Not only are tumor suppressor effects lost, but uncontrolled cell growth is promoted, leading to increased cell division frequency and concomitant increases in mutation rate, and thus further cancers. As a result, an individual patient's resistance to available treatments (e.g., cancer treatment, antiviral therapy) also must be taken into account when determining an effective therapy regimen.

Drug resistance, or therapy resistance, can be determined by phenotypic testing, genotypic testing, or by a combination thereof. Drug resistance, or therapy resistance, is generally determined by two main methods, namely phenotypic testing and genotypic testing, or by a combination thereof. Phenotypic testing directly measures the actual therapy resistance of a patient's malignant or infected cells to a particular therapy or therapies (generating, for example, a concentration of that drug which results in a 50% inhibition of virus growth, i.e., the IC50). The phenotypic testing measures the ability of a virus, for example, to grow in the presence of various drugs. Genotypic resistance testing (sometimes called genotyping) examines the genetic material of the cell or virus to detect the presence of specific genetic mutations or patterns of mutations in the gene or genes of interest that confer resistance to a certain therapy or therapies. Genotyping can be more rapid and less expensive than phenotyping, but may be more difficult to accurately interpret, due to the hundreds of mutations involved, for example, in HIV or p53 oncogenesis.

Although phenotypic testing is believed to be a more comprehensive and accurate assessment of therapy resistance than genotypic testing, phenotypic testing can take longer and may generally be more expensive than genotypic testing. Compared with phenotypic testing, genotypic testing has advantages, including the relative simplicity, low cost, and the speed with which the test can be performed. Currently, genotypic interpretation has predominantly been applied to determining resistance of a virus, e.g., HIV, or mutations in a viral strain to a therapy. In a further development this analysis can be performed using the approach of virtual phenotyping (e.g. VirtualPhenotype, PCT/EP01/04445), wherein the sequence of an etiologic agent is compared to sequences present in a database. The corresponding phenotype can be calculated based on the phenotypic data of the similar sequences.

In addition, a therapy can be less effective or ineffective in an individual because of allelic variations at genes important for the action of a drug. This allelic variation can mean variation at the drug target but also at genes influencing the drug pharmacokintics and pharmacodynamics. Genes which metabolize the drug or receptors influencing the distribution of said drug.

Therefore, because of the importance of maintaining an effective MEC in order to avoid the development of disease resistance, and the need to consider an individual patient's resistance to known therapies in the calculation of optimal dosage of a therapy regime for that patient, there exists a strong need in the art for a single therapeutic procedure to aid doctors with optimizing treatment of these diseases. There also exists in the art a strong need for individualized therapies and optimization of these therapies for individual patients. This need is particularly strong in view of the plasticity the drug response of diseases such as virus infections and malignancies. This optimization should be adaptable to single drugs as well as to combinations of drugs and treatment regimens, and should provide a model with inputs for actual individual patient data as well as overall population data from patients (such as from clinical trials), in order to assess for all known therapies whether plasma levels remain above the MEC throughout therapy on a patient by patient basis.

In the art individual methods are disclosed to determine resistance (e.g. Antivirogram®), to determine the concentration of agents in a biological sample (e.g. high pressure liquid chromatography, mass spectrometry) and to model the pharmacokinetics of drugs administered to individuals. Karlsson M O, Sheiner L B., J Pharmacokinet Biopharm 1993,21:735-750; Mandema J W, Verotta D, Sheiner L B., J Pharmacokinet Biopharm 1992,20:511-528; Thomson A H, Whiting B., Clin Pharmacokinet 1992,22:447467; Wakefield J, Racine-Poon A., Stat Med 1995,14:971-986; Rosner G L, Muller P., J Pharmacokinet Biopharm 1997,25:209-233; Bennett J E, Wakefield J C., J Pharmacokinet Biopharm 1996,24:403-432. Though these methods provide information on either variable, the individual parameters allow limited managing patient treatment. For instance, the drug level in the circulation will not provide evidence regarding the occurrence of resistance. The need for additional data apart from either drug monitoring or RNA testing in the follow-up of HIV therapy was described by Durant and coworkers (AIDS, 2000, 14, 1333-1339). This group linked the RNA levels to the plasma drug concentrations. However, this group did neither use population based modeling, nor phenotypic data, nor the combination thereof to evaluate drug effectiveness. Therefore, in order to design a therapy for diseases such as cancer and retroviral infections, disease states in which resistance displays a critical role, an integrated approach combining resistance testing, bio-analysis and pharmacokinetic modelling is needed to provide a patient specific therapy management. This integrated approach is the subject of the instant invention.

The present invention adds to the art a combination of a bio-analytical method with population based modeling to determine a patient specific measure of therapy exposure, and a resistance determination. The combination of the resistance and patient specific pharmacokinetic parameters provides a single measure to manage therapy. This single variable provides the treating physician with a measure of therapy efficacy and to draw conclusions on an patient specific basis for either drug dosages and resistance patterns.

### SUMMARY OF THE INVENTION

The present invention relates to methods of measuring the efficacy of at least one therapeutic agent comprising a combination of a patient's exposure to a therapy and resistance data. For example, in one embodiment, the invention relates to a method of measuring the efficacy of at least one therapeutic agent comprising: determining a pharmacologic exposure either using a measured or predicted population pharmacokinetic model for said at least one therapeutic agent; determining resistance of an etiologic agent towards said at least one therapeutic agent; determining the inhibitory quotient for said at least one therapeutic agent based on said pharmacologic exposure and said resistance, and using said inhibitory quotient to determine efficacy of said at least one therapeutic agent. In one embodiment, the methods of the invention further comprise the use of a bioanalytical method to obtain an actual concentration of at least one therapeutic agent in a patient. The inhibitory quotient may also, for example, be normalized. In one embodiment, the population pharmacokinetic model for use in any of the embodiments of the invention may be an optimized population pharmacokinetic model.

In one embodiment, the inhibitory quotient used in practicing any aspect of the invention may, for example, be determined by a method comprising:

a) obtaining an actual concentration of at least one therapeutic agent in a patient at a given time using a bionalytical method;

b) calculating a theoretical concentration of said at least one therapeutic agent in said patient at said time using a first population pharmacokinetic model;

c) obtaining a difference by comparing the theoretical concentration of said at least one therapeutic agent with the actual concentration of said at least one therapeutic agent in a patient;

d) minimizing the difference by changing at least one parameter in the first population pharmacokinetic model in order to generate an optimized population pharmacokinetic model;

e) obtaining resistance data from said patient;

f) determining the inhibitory quotient for said at least one therapeutic agent based on said optimized population pharmacokinetic model and said resistance. The method may further comprise the step of normalizing the inhibitory quotient.

The inhibitory quotient, may, for example, be used to optimize at least one of a therapeutic agent regime, including, but not limited to the choice of therapeutic agent, including combinations of therapeutic agents, and the dosage of a therapeutic agent.

The invention encompasses any method or methods of generating resistance data, whether based on genotype, phenotype, or some combination thereof.

The present invention also relates to methods of optimizing at least one therapeutic agent regime for at least one patient comprising a combination of a pharmacokinetic model and resistance data. For example, in one embodiment, the invention relates to a method of optimizing at least one therapeutic agent regime comprising: determining a pharmacologic exposure using a population pharmacokinetic model for at least one therapeutic agent; determining resistance of an etiologic agent towards said at least one therapeutic agent; determining the inhibitory quotient for said at least one therapeutic agent based on said pharmacologic exposure and said resistance, and using said inhibitory quotient to optimize said at least one therapeutic agent regime. In one embodiment, the methods of the invention further comprise the use of a bioanalytical method to obtain an actual concentration of at least one therapeutic agent in a patient. The inhibitory quotient may also, for example, be normalized.

The present invention also relates to methods for obtaining a dosage regime for at least one therapeutic agent for at least one patient comprising a combination of a pharmacokinetic model and resistance data. For example, in one embodiment, the invention relates to a method for determining a dosage regime for at least one therapeutic agent comprising: determining a pharmacologic exposure using a population pharmacokinetic model for at least one therapeutic agent; determining resistance of an etiologic agent towards said at least one therapeutic agent; determining the inhibitory quotient for said at least one therapeutic agent based on said pharmacologic exposure and said resistance, and using said inhibitory quotient to determine a dosage regime for at least one therapeutic agent. In one embodiment, the methods of the invention further comprise the use of a bioanalytical method to obtain an actual concentration of at least one therapeutic agent in a patient. The inhibitory quotient may also, for example, be normalized.

The present invention also relates to methods for providing advice to a physician regarding at least one therapeutic agent for at least one patient comprising a combination of a pharmacokinetic model and resistance data. For example, in one embodiment, the invention relates to a method for providing advice to a physician regarding at least one therapeutic agent for at least one patient comprising: determining a pharmacologic exposure using a population pharmacokinetic model for said at least one therapeutic agent; determining resistance of an etiologic agent towards said at least one therapeutic agent; determining the inhibitory quotient for said at least one therapeutic agent based on said pharmacologic exposure and said resistance, and using said inhibitory quotient to provide advice to a physician regarding at least one therapeutic agent for at least one patient. In one embodiment, the methods of the invention further comprise the use of a bioanalytical method to obtain an actual concentration of at least one therapeutic agent in a patient. The inhibitory quotient may also, for example, be normalized.

The present invention also relates to methods for providing a report regarding at least one therapeutic agent. For example, in one embodiment, the invention relates to a method for providing a report comprising: determining a pharmacologic exposure using a population pharmacokinetic model for said at least one therapeutic agent; determining resistance of an etiologic agent towards said at least one therapeutic agent; determining the inhibitory quotient for said at least one therapeutic agent based on said pharmacologic exposure and said resistance, and providing a report comprising at least one entry chosen from the inhibitory quotient and information derived from the inhibitory quotient. In one embodiment, the methods of the invention further comprise the use of a bioanalytical method to obtain an actual concentration of at least one therapeutic agent in a patient. The inhibitory quotient may also, for example, be normalized. The invention also includes, for example, a report comprising the inhibitory quotient.

In another embodiment, the invention relates to a computer system comprising at least one database comprising at least one inhibitory quotient for at least one patient. The at least one inhibitory quotient may, for example, be a normalized inhibitory quotient.

In another embodiment, the invention relates to a method of identifying at least one therapeutic agent effective against at least one etiological agent comprising: determining a pharmacologic exposure using a population pharmacokinetic model for said at least one therapeutic agent; determining resistance of said etiologic agent towards said at least one therapeutic agent; determining the inhibitory quotient for said at least one therapeutic agent based on said pharmacologic exposure and said resistance, and using said inhibitory quotient to identify at least one therapeutic agent effective against at least one etiological agent. In one embodiment, the methods of the invention further comprise the use of a bioanalytical method to obtain an actual concentration of at least one therapeutic agent in a patient. The inhibitory quotient may also, for example, be normalized.

In a further embodiment, the invention relates to a method of identifying toxic effects of at least one therapeutic agent comprising: determining a pharmacologic exposure using a population pharmacokinetic model for said at least one therapeutic agent; determining resistance of an etiologic agent towards said at least one therapeutic agent; determining the inhibitory quotient for said at least one therapeutic agent based on said pharmacologic exposure and said resistance, and using said inhibitory quotient to identify toxic effects of the least one therapeutic agent. In one embodiment, the methods of the invention further comprise the use of a bioanalytical method to obtain an actual concentration of at least one therapeutic agent in a patient. The inhibitory quotient may also, for example, be normalized.

The invention further relates to systems, computer program products, business methods, server side and client side systems and methods for generating, providing, and transmitting optimal dosage regimens for an individual patient.

Both the foregoing general description and the following detailed description are exemplary and are intended to provide further explanation of the invention as claimed.

### BRIEF DESCRIPTION OF THE DRAWINGS

The accompanying drawings provide a further understanding of the invention and are incorporated in and constitute a part of this specification. The drawings, together with the description, illustrate various embodiments of the invention. In the drawings:

FIG. 1 is an exemplary graph of the concentration in plasma as a function of time;

FIG. 2 is an exemplary flow chart for optimizing a therapy, in accordance with the methods of the invention;

FIG. 3 is an exemplary representation of a system environment in which the features and methods of the invention may be implemented;

FIG. 4 is the relationship between amprenavir NIQ and change in viral load at week 24. Circles are actual values and the line is the fitted value from the sigmoidal Emax model.

### DETAILED DESCRIPTION OF THE INVENTION

These terms as used herein are defined as follows:

“Bioanalytical method” or bioanalytical testing means any analytical technique known in the art to determine the presence and/or the amount or concentration of a therapy in a patient sample. Techniques include, but are not limited to, high performance liquid chromatography, mass spectrometry, LC-MS, radioimmunoassay, enzyme linked immunosorbent assay, and other techniques known in the art.

A “biological sample” is any material obtained from a patient which contains an etiological agent amenable to therapy resistance testing. Some examples are saliva, semen, breast milk, blood, plasma, feces, urine, tissue samples, cells in cell culture, cells which may be further cultured, etc. For example, in a patient infected with HIV, any biological sample containing virus may be used. For a cancer patient, a sample would include all of the above, and tumors, biopsy tissue, etc. from which the sequence of p53 could be determined.

“Clinical data” may include previously recorded patient data, including genotypic variation or patterns with specific therapy sensitivities, data from phenotype-genotype relational databases, 50% inhibitory concentrations and minimum effective concentrations of various therapies, known drug-drug interactions, indications, or contraindications, etc. This clinical data may be generated on-site, off-site, or may be obtained from public databases or journals, or forwarded by researchers in the field.

A “communication channel” is any channel which allows communication between different people, computers, or locations, i.e., telephone lines, wireless networks, computer networks, public networks (such as the Internet), private networks (such as an intranet), satellite-based networks, manual entry of data into a common database, etc. This communication channel may be digital or analog, real time or delayed, and one way or two way, or any combination or combinations thereof between the different entities.

The term “doctors” or “physicians” is understood to include any professional person authorized or trained to treat or take patient data and/or samples. Such persons include but are not limited to clinicians, health care workers, nurses, technicians, etc.

“Dosage” includes the size, frequency, formulation, comedication, and number of doses of at least one therapy to be given to a patient. This also includes newly prescribed therapies and/or therapies, both singly and in combination and is irrespective of the way of administration.

“Resistance” or “therapy resistance” includes any condition by which the cells, etiologic agent or patient respond or adapt to a therapy.

An “etiological agent” is a disease producing agent. Examples of rapidly mutating etiological agents are viruses such as retroviruses, and cancer causing genes or gene mutations such as those found in p53 and other oncogenes. Other agents include bacteria, viruses, prions, algae, fungi, and protozoa.

“Genotypic resistance” comprises changes in the genome of a cell, virus, or diseased cell associated with the resistance to a therapeutic agent or therapy. A diseased cell includes, but is not limited to, cells infected by a virus, or a bacterium, and cells with an altered phenotype by proliferation, inflammation or degeneration.

“Genotypic testing” analyzes part or all of a genetic sequence. This method may include full or partial genomic sequencing by all known means, and may be correlated with phenotype. One such method is the Virtualphenotype® (PCT/EP01/04445).

“HIV” is the human immunodeficiency virus, which is a retrovirus and of which different species are currently known. A retrovirus includes is any RNA virus that utilizes reverse transcriptase during its life cycle.

50% inhibitory concentration, or IC₅₀, is the amount of a substance required to inhibit growth in 50% of cells or organisms in vitro.

“Inhibitory quotient”, IQ, is a ratio of a measure of therapy exposure and a measure of viral susceptibility to that therapy. For example, IQ is the C_{trough} divided by the IC₅₀ for a particular therapy.

A “patient” is any organism, particularly a human or other mammal, suffering from a disease, in need or desire of treatment for a disease, or in need of testing or screening for a disease. A patient includes any mammal, including farm animals or pets, and includes humans of any age or state of development.

“Patient data” includes, but is not limited to, age, gender, weight, height, allergies, other therapies, physical condition, diseases state(s), medications currently being taken, disease status or progression, etc.

“Population pharmacokinetic model” or a pharmacokinetic model predicts an individual plasma concentration of a therapeutic agent using a set of mathematical equation. An “optimized” population pharmacokinetic model is a model that has been adjusted to minimize the difference between at least one data point in the model and at least one actual measurement from a patient. The pharmacokinetic model which describes the drug's behaviour in an organism can be chosen out of variety of models known to the person skilled in the art, including, but not limited to, models based on one compartment, two or more compartments, and using either zero order, first order second order or higher order kinetics. The model may be a predicted model, wherein the model is chosen based on data known in the art for a therapy. Alternatively, the model may be measured by analyzing patient sample and determining the pharmacokinetic model thereon (measured model).

For example, based on literature data and/or drug concentration determinations in patient indications for a model may be provided. A model may allow one to predict or estimate parameters required, e.g. C_{trough}. Patient parameters may also be included in the model, e.g. age, gender, weight, body mass index (Bayes approach). In one embodiment, this combination of data and mathematic equations allows the prediction of parameters including the dosage regimen needed to obtain a certain drug concentration.

“Pharmacologic exposure” is the extent to which a patient is exposed to a therapy. A measure of exposure is, e.g. C_{trough} and area under the curve (AUC).

“Phenotypic resistance” comprises fold-resistance compared to a reference of a cell, virus, or virally infected cell to a tested therapeutic agent or therapy, specifically, traits that can be observed. “Phenotypic testing” is a testing method that obtains this trait of, for example, a cell line or virus. One such method is the high throughput viral screen Antivirogram® (Virco, Belgium; WO97/27480; U.S. Pat. No. 6;221,578).

“Etiological agent” includes any agent which causes disease in a patient. Some examples include, but are not limited to viruses, particularly HIV, bacteria, and mutations associated with malignancies, such as p53.

A “therapeutic agent” is a drug, pharmaceutical, antiviral, anticancer, antifungal, or other compound or composition useful for the treatment of a disease.

“Therapeutic agent regime” is the course of action or use of a therapeutic agent or combination of therapeutic agents in treating a patient including, for example, at least one of dosage, schedule of administration, choice and/or combination of therapeutic agents.

“Therapy” is the treatment of any disease or abnormality, medical treatment of a disease by specified means, such as drugs, treatments, or any procedure to ameliorate a disease. “Therapy resistance,” as used herein, pertains to the capacity of resistance, sensitivity, susceptibility, or effectiveness of a therapy against a disease.

“Trough level” C_{trough} is the lowest concentration of a drug in a patient sample upon the course of therapeutic agent regimen.

“Therapy effectiveness” means having the ability to delay progression of at least one disease and/or to alleviate at least one disease.

In one embodiment, one objective of the development of population pharmacokinetic models for each therapeutic agent is to be able estimate individual pharmacokinetic parameters during therapy using one or more plasma concentrations measured at any time point after therapy intake and having information on the dosage regimen and the time after the last drug intake.

Previous research has attempted to navigate effective dosages of therapeutic agents to challenge rapidly changing etiologic agents. While the broad approach of population pharmacokinetics (usually defined as the change in time of the concentration or nature of therapeutic agent(s) in groups of patients having similar characteristics) is a technique of long standing (see T. M. Ludden, J. Clin. Pharmacol. 28:1059-1062 (1988)), it fails to take into account a large amount of inter-, and even intra-, patient variability, ultimately contributing to therapy failure. In diseases such as AIDS, therapy failure leads to the development (and possible eventual dissemination into the population) of therapy resistant virus strains. Since neither constant therapy monitoring nor completely population-based pharmacokinetic approaches solves all of these inherent shortcomings, a system and method for optimizing therapy is needed.

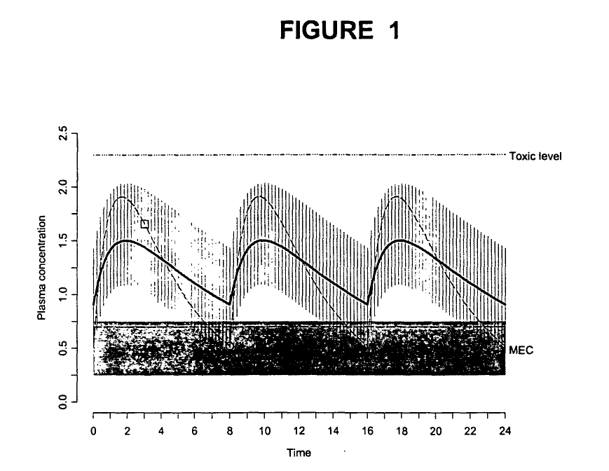

The problem can be best outlined on the basis of an example. Suppose a large group of HIV-infected patients receive the same antiretroviral therapy in the same dose three times daily. The average plasma concentration-time profile of the therapy in the patient population may look as shown in FIG. 1 (bold line). However, due to the inter-individual variability of pharmacokinetic processes (absorption, distribution, elimination), individual plasma concentration-time profiles may substantially differ from the typical profile, as exemplified by the dotted line. A plot of all individual plasma concentration-time profile may cover a range marked by the vertical bars. While individual patient MECs (dashed horizontal line gives an example) may overlap with individual plasma concentration-time profiles or the average plasma concentration-time profile, they may cover an area as broad as the grey area FIG. 1. As a consequence, if the therapy concentration in a patients drops below their MEC resistance may result.

Because of the importance of maintaining an effective concentration to avoid the development of disease resistance, and the need to consider an individual patient's resistance to known therapies in the calculation of optimal dosage for that patient, there exists a need in the art for a therapeutic procedure to aid doctors when optimizing treatment of these diseases.

In one embodiment, the present invention, avoids previously known pitfalls in the art by combining techniques and reiterating obtained data into a model, in order to refine the overall model by reducing errors and to generate an optimized pharmacokinetic model. This optimized pharmacokinetic model is able to correspond to an individual patient at a given time, and may be adjusted to correspond to future points in time. In another embodiment, the methods of the invention may be adaptable to single therapies as well as to combinations of therapy regimens and may provide a model with inputs for actual individual patient data as well as overall population data from patients or individuals (such as from clinical trials), in order to assess for at least one therapy whether plasma levels remain above the MEC throughout therapy on a patient by patient basis.

In one embodiment of the invention, the models of the present invention may change with time according to the patients' disease progression, new or discontinued drug therapy or sensitivity, etc. Systems and methods consistent with the invention may combine at least one bioanalytical method for measuring actual drug concentration in a patient at a given time, resistance data of the individual patient's etiological agent, and a first population pharmacokinetic model which may include any relevant covariates. In one embodiment, the first pharmacokinetic model may include as much individual patient data relevant to treatment as possible to generate dosage(s) for all drug(s) which will maintain a desired trough level, above the MEC, for each drug in each patient throughout the dosage regimen, whether or not such drugs are currently administered to the patient.

The systems and methods of the invention may also, for example, include a database corresponding to the data collected and generated from combined first pharmacokinetic models and/or from combined optimized pharmacokinetic models. This database may include a relational genotype/phenotype database. In a further embodiment, a neural network or computerized platform may also be provided that learns from the patterns in the data collected and generated.

In one embodiment of the invention a bioanalytical method is used in the optimization of the pharmacokinetic model. A bioanalytical method that may be used in the present invention includes, but is not limited to, liquid chromatography with mass spectrometry (LC-MS). An example thereof is a liquid chromatography and mass spectrometry assay system currently available from Virco (Mechelen, Belgium), VIRCO plasmagram™. Moreover, any other bioanalytical method or methods that provide(s) a quantitative measurement of an actual concentration of at least one administered drug may be used in the practice of the invention, though bioanalytical methods which provide a quantitative measurement of all known drugs in one or two procedures in a short amount of time would provide greater efficiency than methods which require longer times and/or more steps. One of skill in the art would realize that in addition to the above method, other bioanalytical methods might also be used, such as straight or reverse phase liquid chromatography (high pressure or ambient pressure), gas chromatography, FPLC, preparative chromatography, gel chromatography, ion exchange chromatography, etc., and by detecting with any known detection method, such as fluorescence, UV-vis, IR, NMR, two dimensional multi-wavelength detection, etc.

For example, in one embodiment, a bioanalytical method may be combined with at least one first pharmacokinetic model in order to optimize individual therapy. Comparison of the theoretical concentration from the first pharmcokinetic model and the actual concentration is a measure of the accuracy of the first pharmacokinetic model. The difference between the theoretical concentration and the actual concentration may then be minimized by changing at least one parameter in the model. Examples of such parameters include any individual patient data, volume of distribution, absorption rate constant, elimination rate constant, etc. In one embodiment, when the difference is minimized, the pharmacokinetic model is optimized for that patient at that time.

In one embodiment, the optimized pharmacokinetic model may be used in which at least one of three different types of variation and their associated errors are checked and minimized: (1) intra-individual variation, where a single patient's parameters may change over time (this includes measurement and sampling errors); (2) inter-individual variation, where an individual patient's parameters differ from the calculation based on previous research and experience; and (3) residual errors, where the theoretically predicted drug concentration differs from the actual measured blood drug concentration errors. The invention may, for example, address all three sources of error by iterative use of the pharmacokinetic model. The methods of the invention may also be encompassed in a database, a neural network relating to the database, and/or by the combined pharmacokinetic model generated from previously collected and iterated patient data (including data from previously conducted clinical studies). In one method of the invention, a neural network is used to obtain resistance data from genotypic data. In another embodiment, a neural network is used to refine the final pharmacokinetic model in order to minimize the difference between the theoretical drug concentration and the actual concentration.

The methods of the invention may also provide, for example, the optimization of therapy for a disease such as cancer and/or retroviral infections (including HIV infections in humans or other mammals). The invention also provides a method of designing a therapy for a patient, and a method of prescribing a therapy for a patient, including making recommendations for drugs and/or combinations of drugs not yet proscribed for that patient.

Population Pharmacokinetic Modeling

Population pharmacokinetic modeling is well known in the art. Karlsson M O, Sheiner L B., J Pharmacokinet Biopharm 1993,21:735-750; Mandema J W, Verotta D, Sheiner L B., J Pharmacokinet Biopharm 1992,20:511-528; Any population pharmacokinetic model known in the art is applicable in the methods of the invention. In one embodiment of the present invention, the concentration data obtained by bioanalysis of human blood samples drawn from a patient is used to develop a population pharmacokinetic model. Other information which may be used in such a model includes, but is not limited to information regarding dosage regimen (dose, dosing frequency, therapy formulation, time of administration etc.), the associated sampling time, co-medication, and patient-specific information.

In one embodiment, a structural pharmacokinetic model may be used in the methods of the invention, which describes the concentration-time course of a therapy. The data will determine which structural pharmacokinetic model may be used to mathematically describe the observed concentration-time courses.

A population pharmacokinetic model may describe both the pharmacokinetics of a therapy in an ‘average’ patient and the variability of certain parameter values in the patient population.

In population pharmacokinetic modeling, the observed therapy concentrations in the blood may be subject to three types of variability. These are the inter-individual and inter-occasion variability in the pharmacokinetic parameters, and a residual intra-patient, variability. The residual variability originates from error in the bio-analysis, misspecification of the time after the last drug intake, model misspecifications etcetera. The inter-occasion variability of model parameters can originate from several causes, such as variability in hepatic metabolism, increased heart rate, increased water retention etcetera. Inter-individual variability of pharmacokinetic parameters also originates from several sources, like the individual's composition of metabolizing enzymes, protein composition of the blood, and many others.

A population pharmacokinetic model may comprise covariates that explain variability of the parameter values. For example, the bodyweight of the patient may be predictive for a certain pharmacokinetic parameter value for that patient. In one embodiment, the developed model may be used to predicted pharmacokinetic parameter values of an individual patient using Bayesian methods. The obtained parameter values may, for example, be used to predict the concentration-time course of the drug in that particular patient.

In most population based model, the principal variables are dependent on the model used. For example, if a one-compartment model is used, one of the variables may concern the distribution volume. Since it is difficult to sample a whole patient population 24 hours a day, a limited set of sample data is usually available for each patient. However, the higher the number of patients the better the estimate of the different pharmacokinetic variables. In one embodiment, using a given a set of data which accurately characterizes the population of interest, the population pharmacokinetic variables can be readily estimated using software like NONMEM. In another embodiment, the data should consist of a sufficient number of patients to characterize the pharmacokinetic variability which exists in the population. This may include deciding which patients to include to cover the natural variability. For example, one may include patients in a broad range of weight, age, renal function.

The NONMEM model, for example, provides a quantitative view of the influence of various factors including pathological and physiological factors on the pharmacokinetics of the drug i.e. the population pharmacokinetic parameters. Briefly, fractional data from individual patients e.g. a drug level, may be used to derive population pharmacokinetic parameters which may then be used to derive individual patient parameters (via Bayesian approach) again using fractional data (e.g. age, . . . ) from different individual patients. The patient specific parameters may then be used to calculate, for an individual patient, the through concentration or to recalculate the drug dosage to be administered to a patient. In one embodiment, this approach may be used to optimize the therapy regimen of an individual patient. For example, one may apply a Bayesian single compartment model.

Inhibitory Quotient

As described above, the influence of resistance testing or therapeutic drug monitoring on clinical outcome has often been evaluated separately. Integration of the two areas has led to the introduction of a new parameter, the “inhibitory quotient” (IQ) as a potential predictor of clinical outcome.

The IQ refers to a measure of the exposure to a therapy in an individual patient (for example, the minimum concentration, Cₘᵢₙ or C_{trough}) divided by the viral susceptibility to that therapy in the same patient (for example, IC50 or “fold change” of IC50 as compared to wild-type virus, as measured in a phenotypic assay). Other measures of therapy exposure include, but are not limited to, area under the curve, clearance, and distribution volume. In one embodiment, the resistance may be determined via a VIRTUALPHENOTYPE® and the virtual IC50 can be used, e.g., IQ may be referred to as virtual inhibitory quotient (VIQ). As used here, IQ includes VIQ. Theoretically, the IQ or VIQ may be a better measure of resistance because viral resistance is relative to therapy exposure.

In one embodiment, by relating individual drug exposure to the level of resistance of the etiological agent in that same individual, a more accurate prediction of response to that drug may be achieved. For example, patients may have adequate drug levels but their etiological agent is moderately resistant, thus they would fail therapy despite good drug exposure. The IQ provides additional information over either test alone (phenotype or therapy level) and may, for example, provide clinicians a guide for dosage adjustment to achieve the desired drug level that can overcome a resistant etiological agent.

The Normalized Inhibitory Quotient

In one embodiment, the normalized inhibitory quotient (NIQ) is a tool to predict clinical outcome using the concept of the inhibitory quotient. Like the inhibitory quotient (IQ), the normalized inhibitory quotient (NIQ) is a ratio of a measure of therapy exposure and a measure of viral susceptibility to that therapy. However, the NIQ corrects for protein binding and may be expressed as follows:

IQptn=IQ of an individual patient determined, for example, by using the actual trough concentration and the individual susceptibility of an etiological agent to a therapy:

$$IQptn = \frac{trough concentration in the individual patient}{fold change of IC50 of the virus in the patient as compared with wild \text{-} type virus} \qquad ( eqn . 1 )$$

The value of IQptn may then related to the reference inhibitory quotient (IQref), which is the IQ of a patient population. For example, IQref is the mean trough concentration of the therapy as known in the population of patients treated with this therapy or the threshold value for the trough concentration divided by the mean fold change of the IC50 of a wild-type virus (unity per definition) or the cut-off value of the fold change for the normal susceptibility range:

$$IQref = \frac{mean trough concentration in the or population or threshold concentration}{fold change of IC50 of wild \text{-} type} \qquad ( eqn . 2 )$$

Finally, the normalized inhibitory quotient is calculated as follows:

$$NIQ = \frac{IQptn}{IQref} \qquad ( eqn 3 )$$

The NIQ may also be multiplied by 100.

The IQ value provides a direct measure of the success of a patient's therapy. In general, the higher the IQ value, the greater the probability that the therapy is effective. Accordingly, the higher the NIQ, the higher the probability that therapy will be successful. In one embodiment, the NIQ should be around 100%. For example, if the NIQ exceeds 100%, the therapy does not need to be changed. While, if the NIQ is below 100%, therapy should be revised, either by increasing the therapy dosage, or by shifting to a different therapy or a combination therapy. In one embodiment, the IQ and NIQ provide the physician with a single value indicative of the therapy effectiveness.

Thus, once the IQ is known for at least one therapeutic agent, for example, the effectiveness of the at least one therapeutic agent is known and at least one therapeutic regime may be optimized by based on the effectiveness of the at least one therapeutic agent. Also, a dosage regime may be adjusted and/or determined, for example, since once the IQ is known for at least one therapeutic agent, whether or not to increase the dosage of the at least one therapeutic agent is, for example, known.

Adjustment of the Dosage Regimen for an Individual

In one embodiment, a Bayesian model may be used to optimize a population pharmacokinetic model. The concept of Bayesian parameter estimation in the field of therapeutic drug monitoring is known in the art and may be useful in circumstances where drug concentrations are measured during relatively complicated dosage regimens, or where only a few concentration measurements are acceptable. The Bayesian method allows an estimation of a patient's pharmacokinetic parameters, so that therapeutic regimens can be adjusted to achieve specific target concentrations. For this purpose, pre-existing information on population characteristics (means and variances) of pharmacokinetic parameters is used in conjunction with the (limited) concentration-time data of an individual patient. The principle of Bayesian estimation is depicted in flow diagram below.

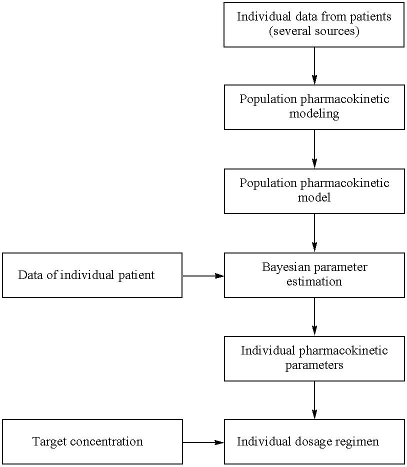

Viral Resistance Typing

In order to obtain effective treatment, the exposure to the drug (trough concentration, AUC, other) should be higher than a certain level. This level is determined by the nature of the etiological population. An indication of the necessary level may be obtained after isolating at least one etiological agent and determining the resistance of at least one etiological to at least one therapeutic agent (Anfivirogram®, VirtualPhenotype™, other).

Generally, phenotypic assays directly measure the ability of a virus to grow in the presence of each drug of interest, where there may be at least one therapy. One technique currently in use, is the ANTIVIROGRAM® (Virco Nev., Mechelen, Belgium), which is an assay for high-throughput analysis of clinical samples that permitts simultaneous detection of HIV phenotypic resistance to both RT and PI (K. Hertogs et al., Antimicrobial Agents and Chemotherapy, 42(2): 269-279 (1998), the disclosure of which is hereby incorporated by reference). In one embodiment, a resistance assay allows an initial estimation of MECs of all known therapies in each patient.

The systems and methods of the invention may be implemented through any suitable combination of hardware, software and/or firmware. Various system components and analytical tools, such as neural networks or artificial intelligence, can be utilized to further optimize a drug therapy for the treatment of a disease. In addition, consistent with the principles of the invention, a database can be generated through a combination of bioanalytical, population pharmacokinetic, and resistance testing methods to provide individualized therapy regimens that can be administered by physicians and the like.

The invention may be embodied, for example, as a method, a data processing system, a computer program product, a business method, or any combination thereof. Although the invention may be practiced without a computer or software-based platform, using a computer or software-based platform may be desirable, given the complexity of the combination and the volume of data of bioanalytical, population pharmacokinetic, and resistance data obtaining methods. Accordingly, the principles of the invention may be implemented as a hardware embodiment, a software embodiment, or any combination thereof, and maybe stored in any computer usable storage medium, i.e., hard disks, CD-ROMs, optical storage devices, magnetic storage devices, etc.

The invention, in one aspect, is described with reference to the accompanying drawings, which include flowchart illustrations of methods and computer program products, as well as system or apparatus diagrams. Each block of the flowchart illustration(s), or combination of blocks in the flowchart illustration(s), can be implemented by computer program instructions. These computer program instructions may be provided to a special purpose computer, a general purpose computer (i.e., a computer not dedicated to the methods of the invention alone), or any other data processing apparatus, to produce a machine such that the instructions, which execute via the processor of the computer or data processing apparatus, create means for implementing the functions specified in the flowchart block or blocks.

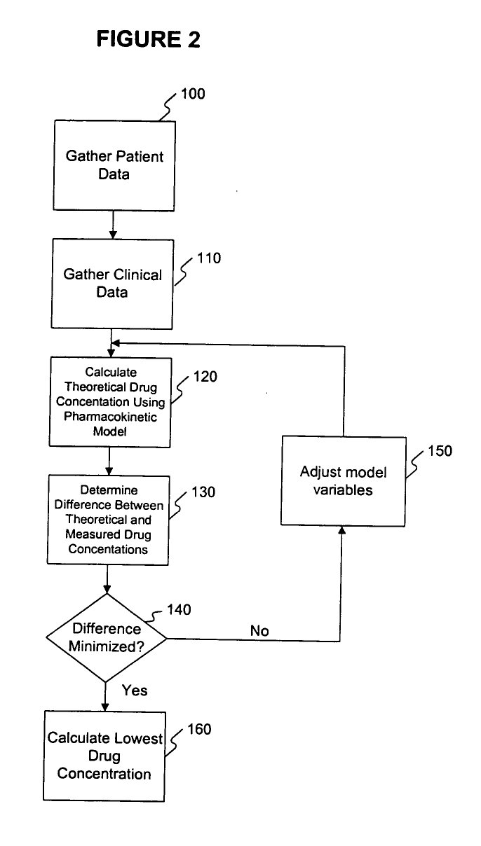

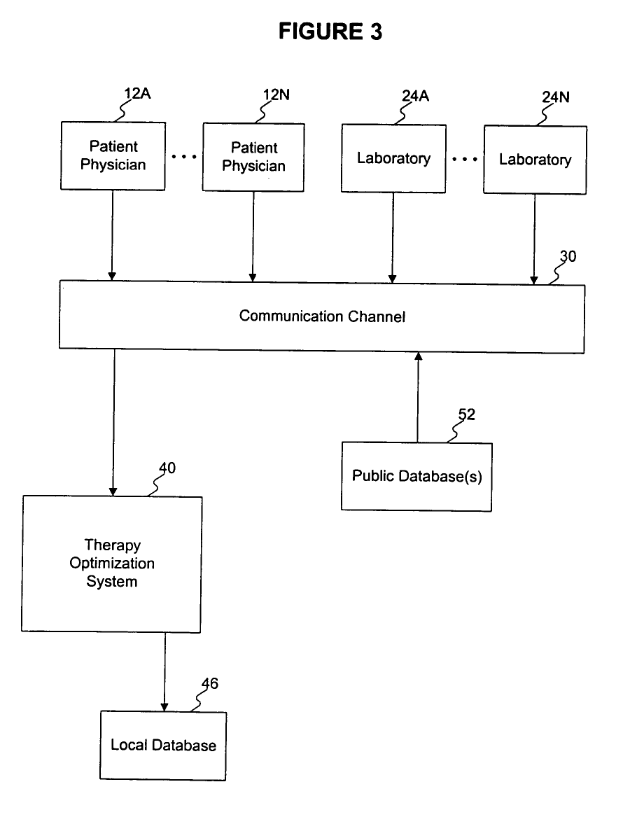

FIG. 2 provides an exemplary flowchart for optimizing drug therapy. In one embodiment, the various steps and operations of FIG. 2 may be performed by the therapy optimization system 40 in the system environment of FIG. 3 to treat a patient diagnosed, for example, with HIV. As indicated above, one of ordinary skill in the art will recognize that the features of the exemplary embodiments can be implemented for the treatment of other diseases, such as cancer, other malignancies, or any disease state mediated by a rapidly mutating etiological agent.

As illustrated in FIG. 2, in one embodiment the process starts with the gathering or collection of patient data (step 100). Patient data may be collected by a physician, a doctor or another entity (including clinicians, health care providers, etc.). The patient data may also include the patient's actual drug concentration for one drug, or as many drugs as the patient is taking at that time, and resistance data that is determined from a patient sample taken at, or close to, that time. In one embodiment, all of the gathered patient data may be stored in a database, such as local database 46 of therapy optimization system 40 (see FIG. 3).

As part of computing an optimized drug therapy, clinical data is also gathered (step 110). As part of this step, therapy optimization system 40 may include data from previous studies (from the same laboratory, and/or from available literature studies) and/or from previous patients with the identified disease or condition. The clinical data, which, for example, may be accessed from local database 46 and/or public database(s) 52, may include data from previous visits from the same patient as a part of the clinical data set. The clinical data may also include data concerning known inter-drug interactions, such as additional sensitivity or synergy, and known drug resistance/phenotype/genotype correlations. Clearly, the order of data collection is irrelevant, and the order may vary from the order described herein. This patient data and clinical data, and any known correlations between, for example, drugs and therapies, may be included in a first pharmacokinetic model.

This pharmacokinetic model may be used to generate a theoretical drug concentration (step 120). The model may also be used to determine a theoretical concentration of any drug currently taken by the patient. One embodiment of the present invention uses a single compartment Bayesian model.

As illustrated in FIG. 2, the theoretical drug concentration, obtained from the pharmacokinetic model, and the actual drug concentration, measured from the patient sample, may then be compared to determine what difference (if any) exists between the theoretical and actual concentrations (step 130). This difference is a measure of model accuracy. Based on this comparison, a determination is made as to whether the difference is minimized (step 140).

If the difference is not minimized (step 140), then at least one parameter may be adjusted in the model (step 150). In one embodiment, the adjustments to the parameters are made so that the difference between the measured and theoretical concentrations is minimized. After adjusting the parameters, the model calculation may be run again to determine a new theoretical concentration (step 120), and the process is iterated again (steps 130-150) until the difference is determined to be minimized (step 140; Yes). In one embodiment, after minimization, the model may be deemed to be a final pharmacokinetic model, optimized for that particular patient at that point in time.

An optimal drug dosage may also, for example, be calculated for that patient at that point in time. In one embodiment, the particular patient's drug concentration should remain above the minimum effective concentration (step 160). In order to accomplish this, the optimized pharmacokinetic model may be used to provide an optimal dosage, by changing the actual dose and/or its frequency.

The information may then be transmitted back to the physician, including recommendations for dosage increases, decreases, or drug changes. Based on the model, which contains information from other clinical studies, and on the patient's resistance profile, an initial estimation may also be made, optimized for that particular patient, as to appropriate dosages for other drugs not yet prescribed to that patient.

FIG. 3 is an exemplary system environment in which the features and methods of the invention may be implemented (for example, the methods as shown in FIG. 2). As illustrated in FIG. 3, a communication channel 30 is provided for facilitating the transfer of data between various system components and entities. These components and entities include one or more physicians 12A-12N who interact with or treat patients (not shown), one or more laboratories 24A-24N, a therapy optimization system 40, and one or more public databases 52.

Communication channel 30 may be implemented through any single or combination of channels that allow communication between different people, computers, or locations. The communication channel may be any system that allows communication between the different entities illustrated in FIG. 3.

Each of the physicians 12A-12N collects data for each patient or patients, wherein such data is submitted for analysis by therapy optimization system 40 and/or laboratories 24A-24N. The patient data gathered by the physicians 12A-12N includes any relevant medical data for that patient and the patient's etiological agent and disease or condition, or at least as much information as is available. As illustrated in FIG. 3, this data can be transferred from each of the physicians 12A-12N to each entity through communication channel 30.

During a patient visit, at least one patient sample may be taken by the doctor or other entity. The patient sample is sent to one of the laboratories 24A-24N to determine data for that patient sample. The patient sample may be obtained at any time, either concurrently or at a different time as a patient visit, and may be provided by a doctor, or may be obtained by another professional at a different time and forwarded to the appropriate site, such as a laboratory. The data from the sample includes the concentration of any drugs currently being taken by the patient for the disease or condition, and the resistance characteristics of the etiological agent. This data may be obtained from a single sample or from multiple samples, depending on the etiological agent and the drug being taken. The drug concentration and resistance data may be provided as part of the patient data to the therapy optimization system 40.

Therapy optimization system 40 may be implemented through any suitable combination of hardware, software and/or firmware. For example, therapy optimization system 40 may be implemented through the use of a personal computer, a working station, a server or any other computing platform. Software or programmed instructions may also be provided for controlling the operations of the computing platform, consistent with the principles of the invention. As illustrated in FIG. 2, therapy optimization system 40 may also include a local database 46 for storing patient data. Local database 46 may also store clinical data or such clinical data may be accessed from one or more public databases 52 by therapy optimization system 40. Consistent with the methods of the present invention, therapy optimization system 40 is configured to optimize and provide a drug therapy for patients treated by physicians 12A-12N. As further described below, the optimization of the drug therapy may be achieved through a combination of bioanalytical, population pharmacokinetic, and resistance testing methods to provide individualized therapy regimens that can be administered to the patient by a physician. The optimized drug therapy may be sent by system 40 to physicians 12A-12N in numerous formats (e.g., written report, electronic file, graphical display, etc.) and may be provided to physicians on fee basis or as a free or ancillary service.

In order to demonstrate embodiments of the invention, an example is presented which describes the optimization of treatment of HIV. For example, the methods of the invention may be useful in regard to both PI's (protease inhibitors) and NNRTI (non-nucleoside reverse transcriptase inhibitors). One of skill in the art will recognize that the present invention can also be used in connection with the treatment of other diseases, and that various modifications can be made (such as the use of a neural network) in order to optimize therapy for individual patients.

### EXAMPLE 1

### Development of a Population Based Pharmacokinetic Method General Outline of an Example Methodology

The data obtained from the quantitative analytical method, i.e., the actual drug circulatory concentration levels, were inputted into a mathematical model. This model was then used to predict the concentration of the drug in the circulation. This prediction, using the model, took into account the dosage, the time between intake and sampling, and other assumptions of the model, i.e., one compartment. Variables were introduced and/or adjusted to close the gap found between the predicted value and the value found through the quantitative analytical model. Validation of the model occurs by approximating these variables as closely as possible.

A classical population pharmacokinetic model may be used to predict an individual plasma concentration of a drug using a set of mathematical equations. One embodiment of the present invention utilized a one-compartment model with absorption. According to this model, at the steady state the concentration of a drug in blood (plasma, serum) can be expressed as follows:

$$\begin{matrix}{C}_{ij} = f ( {P}_{j} , {D}_{j} , {t}_{ij} ) + {ϵ}_{ij} ; \\ f ( {P}_{j} , {D}_{j} , {t}_{ij} ) = \frac{{F}_{j} {D}_{j} {k}_{a , j}}{{V}_{j} ( {k}_{a , j} - {k}_{e , j} )} \begin{matrix}\begin{matrix}□ \\ □\end{matrix} \\ \begin{matrix}□ \\ □\end{matrix}\end{matrix} \frac{exp ( - {k}_{e , j} {t}_{ij} )}{1 - exp ( - {k}_{e , j} {τ}_{j} )} - \frac{exp ( - {k}_{a , j} {t}_{ij} )}{1 - exp ( - {k}_{a , j} {τ}_{j} )} \begin{matrix}\begin{matrix}□ \\ □\end{matrix} \\ \begin{matrix}□ \\ □\end{matrix}\end{matrix}\end{matrix}$$

where Cᵢⱼ is the plasma concentration measured in a patient j at time tᵢ. Dⱼ is a maintenance dose administered with an interdose interval τᵢ. Pᵢ symbolizes a set of individual pharmacokinetic parameters: Vⱼ, k_{a,j} and k_{e,j} (volume of distribution, absorption rate constant and elimination rate constant, respectively). The latter is equal, by definition, to the ratio CLⱼ/Vⱼ, where CLⱼ is an individual value of drug clearance. Fⱼ is a fraction of the dose absorbed after oral administration. It is usually assumed to be equal to one, and thus, estimates of CLⱼ and Vⱼ are actually the ratios of clearance and volume of distribution to the fraction absorbed. εᵢⱼ is a random error reflecting a residual part of the variability in measured concentration not explained by the model. It can often be approximated by the assay error.

k_{a,j}, CLⱼ and Vⱼ may be estimated in each subject and for each drug used to treat this patient. This is a difficult task which normally requires many plasma samples to be drawn from a patient. It may be substantially simplified if we know the distribution of parameters in the patient population:

k_{a,j}=kₐ(θ_{,})+η_{k,j}

Vⱼ=V(θ_{V,j})+η_{V,j}

CLⱼ=CL(θ_{CL,j})+η_{CL,j}

where kₐ, V and CL (without subscript j) is a set of typical parameter values in the patient population. Often one or more typical pharmacokinetic parameters of a particular drug are dependent on patient covariates like body weight or body surface area, age, gender, etc. Individual covariates for the patient j are symbolised by θ_{k,j}, θ_{V,j} and θ_{CL,j} for kₐ, V and CL, respectively. η_{k,i}, η_{V,i} and η_{CL,i} are residual variabilities in individual ka, V and CL, respectively, which remain unexplained after including covariate effects in the model.

The population model of a therapy may be known if typical values of each parameter are known (in the form of equations that relates them to significant covariates, if any) such as residual variabilities in parameters in the patient population and a residual random error in the concentration.

Developing Population Pharmacokinetic Models

Population models for most of the 15 antiretroviral drugs currently used in the treatment of HIV-infected patients have been established: Zidovudine, Lamivudine, Didanosine, Zalcitabine, Stavudine, Abacavir, Nevirapine, Delavirdine, Efavirenz, Saquinavir, Ritonavir, Indinavir, Nelfinavir, Lopinavir, Amprenavir. The models for the remaining drugs may be established using plasma concentrations measured in patients during treatment (therapeutic drug monitoring data). Also, the population models taken from the literature may be verified/validated using the methods of the invention. The population pharmacokinetic program NONMEM based on the approach known as non-linear mixed effect modelling may be used for such modelling.

Since several antiretroviral drugs will be administered to each patient, and he or she may also receive other drugs like antibiotics, antimycotics, etc., an essential aspect of the population model development is searching for drug-drug interactions. If the interaction exists it may be included in a model as a covariate.

Individual Prediction Using Bayesian Feedback

Therapeutic drug monitoring usually assumes taking one or two plasma samples per patient which is not sufficient to find individual estimates of pharmacokinetic parameters of the drugs of interest. The Bayes approach uses both individual plasma concentration measurements and population typical values of pharmacokinetic parameters together with the variability parameters. Bayesian estimates of individual parameters for the patient j, P_{B,j}, are those which minimise the following objective function:

$${OBJ}_{j} = {\sum }_{i}^{} \frac{{[ {C}_{ij} - f ( {P}_{B , j} , {D}_{j} , {t}_{ij} ) ]}^{2}}{{σ}^{2}} - \sum \frac{{[ {P}_{B , j} - P ( {Θ}_{j} ) ]}^{2}}{{Ω}^{2}}$$

where the summation is performed over all concentration measurements and model parameters. σ² is the variance of residual error in the measured concentration of a drug. Ω² is a set of variances corresponding to interindividual variability in parameters (η). θ is a set of all covariates affecting pharmacokinetic parameters.

Having Bayesian estimates of individual parameters it is easy to calculate the trough level by applying the pharmacokinetic model equation again. Moreover, we may also accomplish the inverse task: the calculation of the dose magnitude which will maintain a desired trough level. This can be achieved by solving numerically the following equation with respect to Dⱼ:

C_{trough,j}−f(P_{B,j}, Dⱼ, τ)=0

where C_{trough,j} is in fact a minimum effective concentration as estimated by Antivirogram®. The dose correction according to Bayesian individual predictions is the essence of the Bayesian feedback method of therapy individualisation.

The standard Bayesian feedback method described above sometimes results in too high maintenance dose exceeding the maximum tolerable dose for a given drug. To avoid toxicity one can minimise the difference C_{trough,j}−f(P_{B,j}, Dⱼ, τ) upon condition Dⱼ≦Dₘₐₓ. The interdose interval τ can also be shortened to avoid toxicity, however, more frequent dosing usually leads to poorer compliance. This constrained feedback may substantially reduce the risk of drug-related side effects, however, it may also decrease the therapeutic outcome.

### EXAMPLE 2

### Calculation of Inhibitory Quotient

Two studies demonstrate the use of the IQ or the NIQ for the protease inhibitors lopinavir and indinavir, respectively. In one study in 56 multiple PI-experienced, NNRTI-naïve patients treated with lopinavir plus efavirenz and 2 NRTIs, a correlation was found between the lopinavir IQ and the % of patients with viral load below 400 copies/mL at week 24. The % of patients with viral load below 400 copies/mL at week 24 was 70, 80, and 100% if the lopinavir IQ was <4, 4-15, or >15, respectively. When using the lopinavir trough concentration alone, no correlation with virologic outcome was found.

In another study, a VIQ for indinavir >2 was the strongest predictor of virologic response over 48 weeks in patients who failed an indinavir-containing regimen.10. In this study, patients failing HAART (indinavir 800 mg tid plus 2 NRTIs) were switched to a ritonavir/indinavir 400/400 mg bid regimen, with continuation of the NRTIs during the first 3 weeks. Thereafter, NRTIs were allowed to be switched. Virologic response was defined as having a decline of 0.5 log viral load from baseline, or a viral load below 50 copies/mL. The IQ was a better predictor of response than number of mutations and virtual phenotype fold resistance.

TABLE 1
Summary of available data on the correlations between IQ or VIQ and
clinical outcome.

| | | | | correction | |
|---|---|---|---|---|---|
| Drug | patients | definition of response | cut-off | factor^{#} | ref. |
| lopinavir | 52* | % of patients below 400 copies/mL | IQ > 15 | 0.07 | 9 |
| | | at week 24 | | | |
| indinavir | 24** | % of patients below 50 copies/mL | VIQ > 2 | 0.053 | 10 |
| | | at week 48, or with at least 0.5 log | | | |
| | | drop from baseline | | | |

^{#}correction factor (IC₅₀ of wild-type virus in the presence of 50% human serum) that is multiplied with the fold-change in susceptibility (compared to wild type virus) of the viral strain isolated from the patient
*IQ available for 52 out of 56 patients
**VIQ available for 24 out of 37 patients

### EXAMPLE 3

### Normalized IQ

This example demonstrates how the normalized IQ may provide information regarding efficacy of a therapeutic agent. The first 2 columns of Table 2 represent the trough concentration and fold change of the virus for saquinavir. The next 2 columns represent what a pharmacokinetic model or resistance testing would advise based on these tests alone. The last 4 columns represent what a normalized IQ would advise based on 4 different scenarios for calculating normalized IQ:

Method 1: threshold trough/mean fold change wild-type

Method 2: threshold trough/cut-off fold change

Method 3: mean trough in population/mean fold change wild-type

Method 4: mean trough in population/cut-off fold change

TABLE 2

| Trough in | Fold | Pharm | Virologic | | | Method | Method |
|---|---|---|---|---|---|---|---|
| ng/mL | change | Model | advice | Method 1 | Method 2 | 3 | 4 |
| 500 | 2.0 | Maintain | Sensitive | 125% | 313% | 50% | 125% |
| 200 | 1.0 | Maintain | Sensitive | 100% | 250% | 40% | 100% |
| 500 | 5.0 | Maintain | Resistant | 50% | 125% | 20% | 50% |
| 1000 | 5.0 | Maintain | Resistant | 100% | 250% | 40% | 100% |
| 100 | 0.5 | Increase | Sensitive | 100% | 250% | 40% | 100% |
| 200 | 5.0 | Maintain | Resistant | 20% | 50% | 8% | 20% |
| 50 | 1.0 | Increase | Sensitive | 25% | 63% | 10% | 25% |
| 200 | 2.5 | Maintain | Sensitive | 40% | 100% | 16% | 40% |

In this example, an IQof around 100% provided evidence that the therapy was effective. Furthermore, a decline in IQ indicated that the therapy was becoming less effective, while an increase in IQ may indicate that the drug level is raising to toxic levels.

### EXAMPLE 4

### Optimizing Cancer Therapy

One step for the optimization of cancer therapy is obtaining an actual drug concentration. This may be obtained from any patient material which is amenable to the bioanalytical method chosen. Examples of samples may be solid or liquid, and may be excreted and collected, or may be removed from the patient. Further examples of suitable samples include (but are not limited to) biopsies from bone, muscle, organ, or skin tissue; fecal, saliva, blood, or tear samples; tumor samples from breast, colon, uterine, prostate, or other malignancies.

The resistance data is also collected, wherein the minimum effective concentration (MEC) for at least one drug is determined. This data may come from a phenotypic assay, i.e., from testing of any patient derived product that enables the determination of MEC of at least one drug against the cancer.

Alternatively, or additionally, the resistance data may be obtained from genotypic data. One method is to sequence the genotype, using any one of the methods well known in the art, and to derive resistance data from a genotype/phenotype relational database. The sequencing can be accomplished on all or a part of the genotype, and may focus on a particular oncogene or segment of the genome of particular interest, i.e., on a known tumor suppressor gene such as p53.

The method continues similarly to that used for HIV. A first pharmacokinetic model is used to generate a theoretical drug concentration, which is then compared to the actual drug concentration for that drug in that patient at the specified time. The difference between the two concentrations is then minimized by adjusting at least one parameter in the first pharmacokinetic model. Once the difference is minimized, then the pharmacokinetic model is deemed optimized for that patient. This optimized model is then used in combination with the MEC in order to produce an optimized therapy via dosage recommendations.

### EXAMPLE 5

### NIQ as a Predictor of Virologic Outcome

HIV resistance testing provides information to clinicians regarding the susceptibility of a patient's HIV-1 to a drug compared to susceptibility of a reference strain. Although this has been shown to predict outcome in salvage therapy, it is unable to provide an estimate of whether the patient's drug levels are high enough to inhibit a wild-type or partially resistant strain. Given the wide variability in protease inhibitor concentrations and the common use of pharmacokinetic boosting to achieve higher concentrations, a measure that incorporates both an individual's drug exposure and the viral susceptibility of the infecting virus may be useful in predicting antiviral outcome. This example demonstrates the correlation of NIQ with clinical outcome in treatment-experienced patients.

Methods

Inclusion criteria included: adults (>18 yrs) infected with HIV-1 as determined by ELISA with confirmatory Western blot; a plasma viral burden of >500 RNA copies/ml by bDNA method at a screening visit while receiving a protease inhibitor as a part of combination therapy for the preceding 20 weeks with no protease inhibitor drug change or dose interruption for >3 days in the most recent 12 weeks; a negative serum or urine pregnancy test on the day of enrollment; and a history of no intolerance of ritonavir or nelfinavir. Patients were excluded for pregnancy or lactation, prior exposure to abacavir, amprenavir or efavirenz, concomitant therapy at entry with corticosteroids in other than replacement doses, chemotherapy, or investigational agents, active, untreated opportunistic infection or other major illnesses, malabsorption or other gastrointestinal dysfunction which might interfere with drug absorption or render the patient unable to take oral medication, a history of serious rash (erythema multiforme or Stevens-Johnson syndrome) caused by nevirapine or delavirdine, or concomitant therapy with other drugs that would affect cytochrome P450 metabolism

Patients were enrolled into three parallel treatment groups that included abacavir 300 mg bid, amprenavir 1200 mg bid, and efavirenz 600 mg daily with either low dose ritonavir at 200 mg BID, high dose ritonavir at 500 mg bid, or nelfinavir 1250 mg bid.

Genotyping (VircoGEN II™, VIRCO) and VIRTUAL PHENOTYPE™ were performed on baseline samples. Viral load data were collected at baseline (mean of two pre-therapy samples) and at week 24. Serial pharmacokinetic samples were collected over 12 hours after week 3 for ritonavir-boosted regimens and after week 2 for nelfinavir-boosted regimens.

Amprenavir concentrations in plasma were determined by a validated LC-MS/MS method.

The normalized inhibitory quotient (NIQ) was determined as:

$$NIQ = \frac{IQ patient}{IQ reference}$$

Where the IQ in an individual patient (IQpatient) was calculated as ratio of the patient's trough concentration (Cmin) to the susceptibility of the patient's virus to the drug, expressed as fold change compared to wild type virus (Virtual Phenotype). The IQpt was then related to the reference inhibitory quotient (IQref), in which the mean population trough concentration of the drug from the product label was divided by the cut-off value of the fold change for susceptible viruses.

For amprenavir, nelfinavir, and ritonavir, the concentration 12 hours after dosing was used as the Cmin. For each drug, relationships between viral load change at week 24 and the Cmin, fold-change in resistance, and NIQ were fit to a sigmoidal maximum effect model.

Results

Seventeen patients were available for analysis with pharmacokinetic data, resistance testing, and virologic outcome data at 24 weeks. There were nine patients in the nelfinavir group, four in the low dose ritonavir group, and four in the high dose ritonavir group.

Pharmacokinetics.

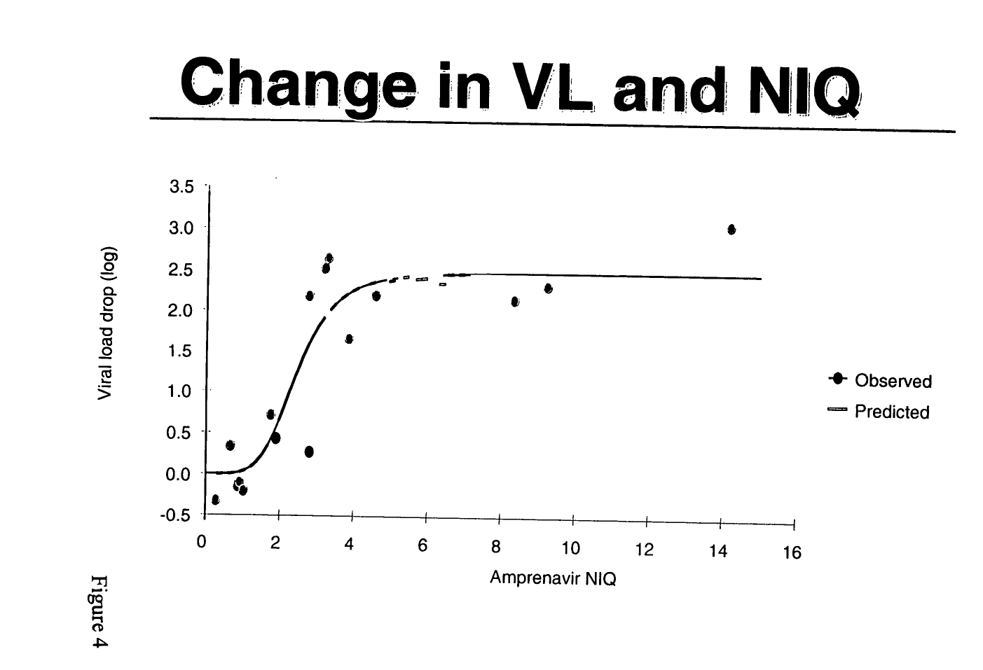

As shown in FIG. 4, the amprenavir (APV) NIQ correlated with outcome at 24 weeks (p<0.05). A decrease in viral load to <400 copies/ml at week 24 was seen in 7/8 patients achieving NIQ >3.0 for APV and 1/9 patients with NIQ<3.0 (p=0.003). Cmin or phenotype alone were less predictive of outcome than the NIQ for APV. Medians and ranges for Cmin, phenotype and NIQs are shown in Table 3. NIQ values for APV were a median (range) of 2.8 (0.3-41.1).

TABLE 3
Individual parameters

| | Cmin | VirtualPhenotype | |
|---|---|---|---|
| Drug | (ng/ml) | (fold-change) | NIQ |
| Amprenavir | 1266 | 4.0 | 2.8 |
| | (264-3453) | (0.6-8.9) | (0.3-41.1) |

All data reported as median (range)

### EXAMPLE 6

### Optimizing Treatment of HIV or Other Virus Infection

A. Overview

The invention of optimizing a therapy as practiced herein for HIV involved a series of iterative steps by which individual patient data and overall population data are combined and interrelated, which produced the most accurate dosage levels for an individual patient. Ultimately, the inventive process is also able to predict accurate individual dosage levels for drugs not yet administered to that patient.

The first step was patient intake, where a complete medical history and description were obtained from each patient. During this intake step, a patient blood sample (either plasma or whole blood) was obtained, wherein the blood sample contained the HIV virus. The intake interview also obtained patient specific data

The blood sample or plasma was divided into aliquots for resistance typing of the HIV virus and quantitative analysis of the drug levels present in the blood. The virus was inactivated prior to being typed. While the viral resistance typing may be accomplished by phenotypic or genotypic analysis, or a combination thereof, one example is as follows:

B. Viral Resistance Typing:

Generally, phenotypic assays directly measure the ability of a virus to grow in the presence of each drug of interest, where there may be one drug, or many drugs. One technique currently in use, Virco's ANTIVIROGRAM® (Virco Nev., Mechelen, Belgium), was the first recombinant virus assay for high-throughput analysis of clinical samples that permitted simultaneous detection of HIV-1 phenotypic resistance to both RT and PI (K. Hertogs et al., Antimicrobial Agents and Chemotherapy, 42(2): 269-279 (1998), the entire disclosure of which is hereby incorporated by reference). Briefly, the assay utilized PCR amplification of a fragment of the viral genome obtained from a patient's blood sample. The amplified fragments and a proviral clone lacking the fragment were electroporated into CD4+, MT4 cells. Successful combination of the provirus and the amplified fragment within the cells resulted in a recombinant virus with a complete HIV-1 genome. This recombinant virus was then grown in cell culture to obtain a recombinant viral stock of known concentration. Susceptibility testing of the recombinant viral stock in the presence of various antiviral agents and a detection system based on green fluorescent protein determined which agents inhibit replication of the recombinant virus as of the time that the sample was taken.

This assay allowed an initial estimation of MECs of all known antiretroviral drugs in each patient. This began the process which enabled (i) selection of most effective combination of drugs to be used in the patient and (ii) therapy optimization using a combination of the patient's drug resistance, bioanalysis of drug levels, and pharmacokinetic modeling.

C. Bioanalysis of Drug Levels

Either concurrently or subsequently, another aliquot of the sample or plasma was analyzed for levels of all drugs currently administered. One assay method for the quantitative determination of plasma levels of all antiretroviral drugs in a sample has been developed and validated and is detailed below. This procedure is advantageous because the sample volume required was as little as 100 microliters, and the complete analytical run could be completed in 15 minutes or less.

This study validated methods for the quantitative analysis of ritonavir (RTV), indinavir (IDV), saquinavir (SQV), nelfinavir (NFV), nevirapine (NVP), delavirdine (DLV), DMP-266 (DMP), amprenavir (AMV), abacavir (ABV), zidovudine (AZT), didanosine (DDI), stavudine (D4T), zalcitabine (DDC) and lamivudine (3TC) in human plasma with LC-MS/MS. This embodiment illustrates a single quantitative analysis method, though any quantitative analytical method known in the art may be used. This quantitative analysis determined the levels of those substances in plasma samples of HIV-patients as a part of therapeutic drug monitoring.

Experimental Methods:

The following data and conditions validated the detection process for one bioanalytical process which may be used according to the invention. The process was based on LC/MS, and its accuracy was confirmed for all relevant storage conditions, quality control parameters, etc. as follows:

HPLC and Mass Spectrometric Conditions

For practical reasons, two different LC-MS/MS methods were applied for quantification of the test substances. The test substances were divided in two groups (group 1 and group 2) dependent on the suitability of analytical methods. For each group of test substances a method was validated.

Group 1 HPLC and MS-conditions (RTV, IDV, SQV, NFV, NVP, DLV, DMP, and AMV):

The LC-MS/MS conditions for the analysis of the test substances in human plasma for Group 1 were as follows.

The HPLC Column and Guard Column were both SYMMETRY C18 50 mm×2.1 mm; dp=3.5 μm (Waters) (except the guard column was 10 mm), and the LC method was run at ambient temperature with a flow rate of 0.3 ml/min. The mobile phase was a gradient of Solvent A: 10/90 methanol/Milli-Q; 2.5 mM ammonium acetate absolute and Solvent B: 90/10 methanol/Milli-Q; 2.5 mM ammonium acetate absolute, according to the table as follows.

| Time [min] | % A | % B | % water | % methanol |
|---|---|---|---|---|
| 0 | 62.5 | 37.5 | 60 | 40 |
| 0.5 | 62.5 | 37.5 | 60 | 40 |
| 0.51 | 31.5 | 68.5 | 35 | 65 |
| 2 | 31.5 | 68.5 | 35 | 65 |
| 4 | 0 | 100 | 10 | 90 |
| 6 | 0 | 100 | 10 | 90 |
| 6.1 | 62.5 | 37.5 | 60 | 40 |
| 10 | 62.5 | 37.5 | 60 | 40 |

Detection: API 300 mass spectrometer (PE-Sciex, Toronto, Canada)

Interface: Turbo Ionspray: positive mode; Temp 400° C.; flow 5000 ml/min

Masses Monitored Period 1:

NVP: 266.8→226.2,

Dwell time: 1350 ms, Pause time: 50 ms

Period 2:

DLV: 457.3→220.9, SQV: 671.3→570.1, IDV: 614.5→421.0, NFV: 568.5→330.0, RTV: 721.5→295.8, DMP: 316.2→243.9, AMV: 506.4→245.1,

Dwell time: 150 ms, Pause time: 50 ms

Split ratio no split Injection volume: 3 μl

Group 2 HPLC and MS-conditions (ABV, AZT, DDI, D4T, DDC and 3TC):

The LC-MS/MS conditions for the analysis of the group 2 test substances in human plasma samples were as follows. The HPLC column was SYMMETRY C18 150 mm×3.0 mm; dp=5 μm, and the guard column was SYMMETRY C18 20 mm×3.9 mm; dp=5 μm, both from Waters Corporation, Milford, Mass., USA). The LC was run at ambient temperature, with a flow rate of 0.4 ml/min. The mobile phase was a gradient of Solvent C: Milli-Q water with 2.5 mM ammonium acetate, and Solvent D: 100 methanol with 2.5 mM ammonium acetate, according to the table as follows:

| T [min] | % C | % D | % water | % Methanol |
|---|---|---|---|---|
| 0 | 70 | 30 | 70 | 30 |
| 6 | 60 | 40 | 60 | 40 |
| 8 | 40 | 60 | 40 | 60 |
| 8.1 | 70 | 30 | 70 | 30 |
| 12 | 70 | 30 | 70 | 30 |

Detection: API 300 mass spectrometer (PE-Sciex, Toronto, Canada)

Interface: Positive Turbo ionspray; Temp 350° C.; flow: 4000 ml/min

Masses Monitored Period 1:

DDI: 229.8→111.9, D4T: 225.2→127.1, DDC: 211.8→111.9, 3TC: 237.0→136.9,

all dwell time: 250 ms, pause time: 50 ms

Masses Monitored Period 2:

AZT: 268.4→127.1, ABV: 287.4→191.0,

all dwell time: 600 ms

pause time: 50 ms

Splitratio approximately 1:2 (flow to the MS about 130 μl/min)

Injection volume: 50 μl

Stock and Standard Solutions

Stock solutions of all test substances of group 1 at 1000 μg/ml (weight corrected for purity) were prepared by dissolving an exact amount of approximately 1 mg of test substances in methanol. Methanol was added to obtain exact concentrations of 1000 μg/ml.

Stock solutions of all test substances of group 2 at 1000 μg/ml (corrected weight for purity) were prepared by dissolving an exact amount of approximately 1 mg of test substances in methanol. Methanol was added to obtain exact concentrations of 1000 μg/ml.

For each test substance, two stock solutions were prepared, one for the preparation of calibration standards (stock solutions 1) and one for the preparation of Quality Control samples (stock solutions 2). The stock and standard solutions (working solutions, K-references and spike solutions) were stored in the freezer at about −20° C.

Calibration Standards

Working solutions containing all test substances per group were prepared by dilution of the corresponding stock solutions 1. The working solutions were used to prepare plasma calibration standards by adding 1 volume of working solution to 10 volumes of plasma. The concentrations of the test substances in the working solutions that were used for validation are outlined in Table 1.

TABLE 4
Test substance concentrations (ng/ml) in plasma calibration standards

| | Calibration standard reference number | | | | | | |
|---|---|---|---|---|---|---|---|
| Name | 1 | 2 | 3 | 4 | 5 | 6 | 7 |
| Group 1 | | | | | | | |
| NYP | 100 | 200 | 500 | 1000 | 2000 | 5000 | 10000 |
| DLV | 82.6 | 206 | 619 | 1858 | 4128 | 8256 | 16512 |
| IDV | 100 | 200 | 500 | 1000 | 2000 | 5000 | 10000 |
| DMP | 100 | 200 | 500 | 1000 | 2000 | 5000 | 10000 |
| RTV | 100 | 250 | 750 | 1000 | 2500 | 7500 | 15000 |
| SQV | 43.7 | 87.4 | 218 | 655 | 1747 | 4368 | 8735 |
| NFV | 50 | 100 | 250 | 750 | 2000 | 5000 | 10000 |
| AMV | 25 | 75 | 225 | 675 | 2000 | 5000 | 10000 |
| Group 2 | | | | | | | |
| DDC | 0.3 | 0.6 | 1.25 | 2.5 | 5 | 7.5 | 10 |
| DDI | 25 | 50 | 100 | 250 | 500 | 750 | 1000 |
| 3TC | 25 | 75 | 200 | 500 | 1000 | 2500 | 5000 |
| D4T | 25 | 50 | 100 | 250 | 500 | 750 | 1000 |
| AZT | 25 | 50 | 100 | 250 | 500 | 750 | 1000 |
| ABV | 50 | 100 | 250 | 750 | 2000 | 5000 | 10000 |

The plasma calibration standards were processed according to the work-up procedure as outlined above.

Quality Control Samples

Spike solutions for group 1 were used to prepare pools of plasma quality control samples for group 1 by adding 1 volume of spiking solution to 10 volumes of plasma. The spike solutions for group 2 were used to prepare pools of plasma quality control samples for group 2 by adding 1 volume of spiking solution to 20 volumes of plasma. The concentrations of the quality control samples for each test substance are given in Table 5.

TABLE 5
Test substance concentration for quality control samples in ng/ml

| Name | Low | Mid | High |
|---|---|---|---|
| Group 1 | | | |
| NVP | 120 | 1000 | 9000 |
| DLV | 99.1 | 1651 | 14861 |
| IDV | 120 | 1000 | 9000 |
| DMP | 120 | 1000 | 9000 |
| RTV | 120 | 1500 | 14000 |
| SQV | 52.4 | 874 | 7862 |
| NFV | 60 | 1000 | 9000 |
| AMV | 30 | 1000 | 9000 |
| Group 2 | | | |
| DDC | 0.36 | 2 | 9 |
| DDI | 30 | 200 | 900 |
| 3TC | 30 | 500 | 4500 |
| D4T | 30 | 200 | 900 |
| AZT | 30 | 200 | 900 |
| ABV | 60 | 1000 | 9000 |

After preparation, the QC-solutions were aliquoted and stored at −20° C. until use. All QCs were processed according to the work-up procedure as outlined in the experimental part.

K-References

K-reference solution per group consisted of a mixture of all test substances in mobile phase at a concentration level of the middle QC.

Validation Procedure

For both groups, three analytical batches were processed. Each batch consisted of:

Duplicate set of calibration standards at each of seven concentrations. One set was analyzed at the beginning of the analytical batch, and one was analyzed at the end of the analytical batch in order to verify the calibration over the time period for sample analysis. The time between the HPLC analysis of the two sets was about 20 hours, which corresponds with the approximate time required for analysis of the QCs and about 100 samples.

Quality Control samples (QCs) at three levels in triplicate.

One plasma blank

K-references

System Performance

The K-references were used to monitor the performance of the LC-MS/MS system. For this purpose a K-reference solution was injected regularly during each analytical batch. The mean peak area and its coefficient of variation were calculated.

Response Function

Peak areas of both sets of calibration standards together were fitted using least squares linear regression. For all test substances the optimal weighing factor was determined.

Sensitivity (LLOQ)

The LLOQ (lower limit of quantification) of the test substances was set at the concentration of the lowest calibration standard.

Precision and Accuracy

The accuracy was shown to be within the calibration range by the following procedure. The regression parameters (slope and intercept) were used to determine the sample concentrations and to recalculate the concentrations of the calibration standards on the regression line (determination of the accuracy within the calibration range). The accuracy was determined as the percentage relative error (RE).

The performance of the method in terms of accuracy and precision was established by analysis of quality control (QC) samples and calculation on the calibration curve in plasma.

For each of the three concentration levels, the within-batch and between-batch precision and accuracy were determined from the results of the QC samples. The within-batch (n=3) and between-batch (n=3 of the mean within-batch determinations) precision were determined as the coefficient of variation (CV) of the mean areas; the accuracy was determined as the percentage relative error (RE). The within-batch and between-batch precision and accuracy were also determined in QCs of which only 55 μl or 27.5 μl was processed.

The absolute recovery was analyzed by the following method. Triplicate QCs at each of the levels were worked-up. Also in triplicate, blank plasma was worked-up. In the last step of the sample preparation procedure, to 100 μl of the extracted blank 100 μl of 5 mM ammonium acetate was added containing the relevant test substances at a concentration of two times the theoretical concentration in end solution. The absolute recovery was calculated by comparison of the peak areas of the QCs with the peak areas of the plasma samples that were spiked after processing the samples.

The matrix effect on the LC-MS/MS analysis was determined by analyzing 6 different batches of plasma at the lowest QC-level. Also, several pools of plasma, obtained from HIV-patients were used for this purpose.

Of each plasma batch, in duplicate blank plasma was processed according to the sample preparation procedures. In the last step of the procedure to 100 μl of the extracted sample 100 μl of 5 mM ammonium acetate was added, containing the relevant test substances at a concentration of two times the theoretical concentration in end solution for the lowest QC-level. The areas of the test substances in these samples were compared with the areas of the test substances in end solution.

Specificity

The identity of the group1 test substances (RTV, IDV, SQV, NFV, NVP, DLV, DMP, and AMV) and the group 2 test substances (ABV, AZT, DDI, D4T, DDC, and 3TC) was demonstrated by the response under the specific MRM conditions of the analyte and by the retention time of the analyte. The absence of interference was verified by processing blank plasma in each analytical batch.

Stability Analyses

a) Freeze/thaw stability: Triplicate QCs at the mid QC level were processed after 1 and 4 freeze/thaw cycles. Each cycle involved at least 4 hours at −20° C. and thawing for 2 hours at >15° C.

b) Stability in human plasma at room temperature and at 4° C. in the dark: Triplicate QCs at the mid QC level were processed directly after thawing and after at least 24 hours storage.

c) Stability in human plasma at −20° C.: Triplicate QCs at the mid QC level were processed at several time-points after preparation. At least a 2-week interval was monitored.

d) Stability in human plasma at 55° C. for 4 hours: Triplicate QCs at the low, middle and high QC level were sent to Virco on dry ice.

Samples are handled according to proper biohazard procedures, i.e., an authorized person in a biohazard lab cabinet unpacked the QC's. The data on the tubes was checked with the data on the accompanying list. New tubes were prepared and identified. The plasma was thawed and transferred into the new tubes. The caps of the tubes were decontaminated with ethanol. The sample was transferred into the incubator and heated at 55° C. for 4 hours. The samples were cooled to room temperature and subsequently stored at −80° C. until they were analyzed. Samples were maintained on dry ice during transfers.

For reference, an additional set of triplicate QCs at the low, middle and high QC levels was sent on dry ice and stored at about −20° C. Hereafter, the QC's were returned together with the heated QCs on dry ice and processed.

e) Stability in end solution at room temperature and at 4° C. in the dark: Triplicate QCs at the mid QC level were processed and analyzed within 8 hours and after at least 78 hours of storage.

f) Stability of stock solutions in solvents at −20° C.: UV spectra of all test substances were measured on dilutions of the stock solutions in DMSO, methanol, or Milli-Q water at several time-points between the preparation of the stock and the end of this validation study. The spectra and the extinction coefficients at the absorption maxima were compared. The absorbance A (1%, 1 cm) was calculated.

While the above method has been quality control validated for a single method, i.e., high pressure liquid chromatography combined with mass spectrometry, any quantitative method which separates, identifies and quantifies the drugs of interest may be used.

Individual MECs and plasma levels of all drugs so obtained are then utilized in and incorporated into a population pharmacokinetic model as described below, making possible the forecasting of optimal individual drug dosage via Bayesian feedback. The optimal dosage is defined as the maintenance dose coupled with the interdose interval which ensures the trough level of each drug remains above the corresponding MEC, but below a minimum toxic level.

D. Population Pharmacokinetic Analysis

The population pharmacokinetic models for each therapeutic drug or antiretroviral compound allowed the estimation of the trough level during therapy for each therapeutic compound, using plasma concentrations measured at any time point after drug intake. This analysis utilizes both the resistance data and the plasma concentrations derived from the initial patient sample, and also incorporates any relevant patient data obtained at intake.

This methodology of utilizing the MECs and plasma concentrations in the pharmacokinetic model may be best explained by way of example. A large group of HIV-infected patients receives the same antiretroviral drug in the same dose three times daily, yielding an overall typical plasma concentration-time profile of the drug for the group as shown by the bold line in FIG. 1. Inter-individual variability of pharmacokinetic parameters gives individual curves which may substantially differ from the typical profile as indicated by the dotted line. If all individual curves are plotted, they would cover the range marked by vertical bars. If individual MECs are shown on the same graph (where the dashed horizontal line illustrates a single example), they will also cover some range, as indicated by shading. Due to cyclic behavior of the drug concentration profiles, the drug level in some of the patients may drop below their MEC, potentially negatively effecting the therapeutic outcome.

The ANTIVIROGRAM® assay (a high throughput, recombinant virus assay which measures the viral susceptibility of a patient sample to all available antiviral drugs) provides an individual MEC, and if a trough plasma level of the drug were known (shown as a circle on the plot), the dosage may be recalculated in a simple way and then modified to get a trough value which exceeds MEC. However, blood samples are usually withdrawn at random times, and often sampling times do not coincide with the time of taking a drug (a square on the plot), precluding the direct calculation of an optimal dosage. However, with a population pharmacokinetic model which includes estimates of pharmacokinetic parameters in a typical patient and of the interindividual variability in these parameters across the patient population, a Bayesian approach will estimate the most probable individual parameter estimates and then the dosages may be adjusted so as to maintain the trough level which exceeds MEC for that particular patient as described.

1. A method of measuring the efficacy of at least one therapeutic agent comprising:
determining an actual concentration of said at least one therapeutic agent;
determining a pharmacologic exposure using a population pharmacokinetic model for said at least one therapeutic agent;
determining resistance of an etiologic agent towards said at least one therapeutic agent;
determining an inhibitory quotient for said at least one therapeutic agent based on said pharmacologic exposure and said resistance, and
using said inhibitory quotient to determine efficacy of said at least one therapeutic agent.

2. The method of claim 1, wherein said inhibitory quotient is a normalized inhibitory quotient.

3. The method of claim 1, wherein the pharmacologic exposure is a trough concentration

4. The method of claim 1, wherein the resistance is derived from a phenotypic determination.

5. The method of claim 1, wherein the population pharmacokinetic model is chosen from a measured population pharmacokinetic model and a predicted population pharmacokinetic model.

6. The method of claim 1, wherein the resistance is determined from a virtual phenotype determination.

7. The method of claim 1, wherein the optimized pharmacokinetic model minimizes at least one error selected from intra-individual, inter-individual, and residual error.

8. The method of claim 1, wherein the resistance data is obtained from a sample chosen from at least one of a plasma sample, a blood sample, a saliva sample, a tumor sample, a tissue sample, and a bodily fluid sample.

9. The method of claim 8, wherein the sample is a virus-containing sample.

10. The method of claim 9, wherein the virus is a retrovirus.

11. The method of claim 10, wherein the retrovirus is Human Immunodeficiency Virus (HIV).

12. The method of claim 8, wherein the sample contains malignant cells.

13. The method of claim 1 wherein the optimized population pharmacokinetic model is optimized using a Bayesian model.

14. The method of claim 1 further comprising determining an optimal dosage for all therapies in series of therapies.

15. The method of claim 1 further comprising entering said inhibitory quotient in a computer database.

16. The method of claim 1, wherein the at least one therapeutic agent is an anti-infectious compound.

17. The method of claim 16, wherein the anti-infectious compound is an anti-retroviral agent.

18. The method of claim 1, wherein the anti-infectious compound is an anti-tumoral agent

19. A method of measuring the efficacy of at least one therapeutic agent comprising:
a) obtaining an actual concentration of at least one therapeutic agent in a patient at a given time using a bioanalytical method;
b) calculating a theoretical concentration of said at least one therapeutic agent in said patient at said time using a first population pharmacokinetic model;
c) obtaining a difference by comparing the theoretical concentration of said at least one therapeutic agent with the actual concentration of said at least one therapeutic agent in a patient;
d) minimizing the difference by changing at least one parameter in the first population pharmacokinetic model in order to generate an optimized population pharmacokinetic model;
e) obtaining resistance data from said patient;
f) determining the inhibitory quotient for said at least one therapeutic agent based on said optimized population pharmacokinetic model and said resistance, and
g) using said inhibitory quotient to determine efficacy of said at least one therapeutic agent.

20. The method of claim 19, wherein said inhibitory quotient is a normalized inhibitory quotient.

21. The method of claim 19, wherein the inhibitory quotient is determined using a trough concentration from the optimized population pharmacokinetic model.

22. The method of claim 19, wherein the resistance is derived from a phenotypic determination.

23. The method of claim 19, wherein the population pharmacokinetic model is chosen from a measured population pharmacokinetic model and a predicted population pharmacokinetic model.

24. The method of claim 19, wherein the resistance is determined from a virtual phenotype determination.

25. The method of claim 19, wherein the optimized pharmacokinetic model minimizes at least one error selected from intra-individual, inter-individual, and residual error.

26. The method of claim 19, wherein the resistance data is obtained from a sample chosen from at least one of a plasma sample, a blood sample, a saliva sample, a tumor sample, a tissue sample, and a bodily fluid sample.

27. The method of claim 26, wherein the sample is a virus-containing sample.

28. The method of claim 27, wherein the virus is a retrovirus.

29. The method of claim 28, wherein the retrovirus is Human Immunodeficiency Virus (HIV).

30. The method of claim 26, wherein the sample contains malignant cells.

31. The method of claim 19, wherein the resistance data is determined by a high-throughput screen.

32. The method of claim 19, wherein the optimized population pharmacokinetic model is optimized using a Bayesian approach.

33. The method of claim 19 further comprising determining an optimal dosage for all therapies in series of therapies.

34. The method of claim 19 further comprising entering said inhibitory quotient in a computer database.

35. The method of claim 19 further comprising the use of the inhibitory quotient to provide advice to a physician wherein said advice is chosen from: choice of at least one of a therapeutic, effectiveness of at least one therapeutic agent and dosage of at least one therapeutic agent.

36. The method of claim 19, wherein the at least one therapeutic agent is an anti-infectious compound.

37. The method of claim 36, wherein the anti-infectious compound is an anti-retroviral agent.

38. The method of claim 36, wherein the anti-infectious compound is an anti-tumoral agent

39. A method of optimizing at least one therapeutic agent regime comprising:
determining a pharmacologic exposure using an optimized population pharmacokinetic model for at least one therapeutic agent;
determining resistance of an etiologic agent towards said at least one therapeutic agent;
determining the inhibitory quotient for said at least one therapeutic agent based on said pharmacologic exposure and said resistance, and
using said inhibitory quotient to optimize said at least one therapeutic agent regime.

40. A method for determining a dosage regime for at least one therapeutic agent comprising:
determining a pharmacologic exposure using an optimized population pharmacokinetic model for at least one therapeutic agent;
determining resistance of an etiologic agent towards said at least one therapeutic agent;
determining the inhibitory quotient for said at least one therapeutic agent based on said pharmacologic exposure and said resistance,
and using said inhibitory quotient to determine a dosage regime for at least one therapeutic agent.

41. A method for providing advice to a physician regarding at least one therapeutic agent for at least one patient comprising:
determining a pharmacologic exposure using an optimized population pharmacokinetic model for said at least one therapeutic agent;
determining resistance of an etiologic agent towards said at least one therapeutic agent;
determining the inhibitory quotient for said at least one therapeutic agent based on said pharmacologic exposure and said resistance,
and using said inhibitory quotient to provide advice to a physician regarding at least one therapeutic agent for at least one patient.

42. A method for providing a report comprising:
determining a pharmacologic exposure using an optimized population pharmacokinetic model for said at least one therapeutic agent;
determining resistance of an etiologic agent towards said at least one therapeutic agent;
determining the inhibitory quotient for said at least one therapeutic agent based on said pharmacologic exposure and said resistance, and
providing a report regarding comprising at least one entry chosen from the inhibitory quotient and information derived from the inhibitory quotient

43. A report comprising a normalized inhibitory quotient.

44. A computer system comprising at least one database comprising at least one inhibitory quotient for at least one patient.

45. A method of identifying at least one therapeutic agent effective against at least one etiological agent comprising:
determining a pharmacologic exposure using an optimized population pharmacokinetic model for said at least one therapeutic agent;
determining resistance of said etiologic agent towards said at least one therapeutic agent;
determining the inhibitory quotient for said at least one therapeutic agent based on said pharmacologic exposure and said resistance,
and using said inhibitory quotient to identify at least one therapeutic agent effective against at least one etiological agent.
